SEQUENCE LISTING (2 sequences)

SEQ ID NO: 1 - DNA, 2517 bp, Artificial Sequence
  homologous recombination region 1
ttttaagaaaattcatccagctgtaaaatatatgcattggttttaaactaaattcttatgcgattttgcttttcagtacaaatacagagacctaactgtacgtgaaactgtcaatgttattactctatacaaagatctcaaacctgttttggattcatatggtgagtttatgcagtaaaaatagcaatttctatactttgagtttactctctttgttaatgattgtaattttttccatttgaggtttgtggaggtttgtggatttttcaattgtgggattgcaccaccgcctaataaaactgttggagggtagcaaattagaagatctaataaaaacatttcatatttctttagttttagttttttatgttaaaaaaaaatactgtacctggatgtggtggctcatttctgtaatcccagcactttgggaggctgaggtgggtggaccactgagcccaggaggttgagaccagcctgggcaatatggtgaaacctcgtctcaacaaaaaaatacttaaagttggcctggtgctcctgtagtcccagctacccaggaggctgaggtggaaggattgcttgagtccaggaggcagtccaggaggcagaggttgcagtgagccgagatcgggccactgcactccagcctgggcaacagagcaagaacctgtctcaaaaaaataaaaaacaaaacctctaacatagaactatgcaaatttaaacctaggaggggatgttaaagataatttagtccatttcccaaagtgttgtctgtagacccttagttatgctgggaacaggggtagaggcagaattctatagttaagcttgagcaacttatggattaaacaaaattgagtttcattgcatgaagatttatcagagcctttagtatgctaatgtgttgtgtatcaccgagacgagcaagaatatattgtataatacatcctaaagttatttaactgctgaacctccctgcccccatagtgtctctttatattttcaggaacacattttgcgaaactatactctggactgtccctttcattttatagatgaggaaaatgttatttaatgtggtctttaattctgtagagtaggtaagcataacagtttgtctctactttctattgagacaaagttgtaaggcaagacaacgcttggagttttccttattttaaaatagtcttttctgtccctaccaaatcctaacataatttcttcaaccctgtctccttgaaaataatatgccagggccgagggaaaacccatgctgctgcttgtccattgtgagtcccttagctctgaaagcaaggaactgaattttgtagctgagactttctaaatttcatttgcttccaaggctttgaaaacattaggaaactggtgaagagaggtgggaagcaacagaggggcaatcagttctgcatttcctgaacaataaagacatgaacccaaagtcctcttccaaacctaggacacgattccttctcatctcagcctaccttatttctgtctgcatactatatgtacaatggtattttgaactacaaaggcctcacattaccaaaattaaagttagtttttaaatggcttcagtggggagaaaaatggttggagctagaattttatagtttttactgcatataaaagaataaatacatttatcaaaactgacaaagactccattataaagtcttgtatagtttcatgtggctggactaagtgtaaatcattgttaacaaatacttttagagtaaacaaagccccaaatttatataaggtggttttcttttttaaaatgcacaaaatcagaatacattgcggtatagcttacattcgtcaacagtaagtaaaataacaaaggtcaagaatgtacagtcgtgcattttgtagtgatagagatatggtctgagaaatgcattatttggcaatttcatcattgagtgtacttagacaaacctagatggtatatcctactacacacctaggttatatggcatagcctgttgctcctaggctacaacctataccagatgttactgtactgcataccataggcagttgtaacacaatggtatttatgtatctaaccgtagaaaagttacagtaaaaatacagtattataatcttatgggaccagtgttcttatgtgcagtccattgtagacccaaacattacacagtagatggctatagttctatgtttttgtactgcacattacaacctttcctctatgggtatagtgttaattccaaattattgttagaaataatagctgtccaatacaaactatgtgccatattaattatagacataaattatagatagaaaaatgtgtgtggtatgagaaatacagattgaaagaaattgtttatatttggctatgaacttttctttttttcattttaatactggctaaggaggctgaggccaggagaccatttgagtccaggagttcaagtccagcccggacaacttagaccccatttctaaaaaaaaaaaaagctgggcatggta

SEQ ID NO: 2 - DNA, 1894 bp, Artificial Sequence
  homologous recombination region 2
gtgcacgcctgtagtcccagctacacggaaggctgaggtgggaggattgcttgagcctagagtttgaggatagcctgcacaacatagcaagaccctgtctccaaaaaacaataataaataaataactgtgagcgactagggataaatgtctgcacatctctaaaatagaaagacagtaggttaacatttagtatggtattgtttgatagtgtttttttgtttgtttgtttgcagtggagttttgctcttgttgcccagactaaagtgcaatgggcacagtctcggctcactgcaacctctgcctcctgggttcaagcaattctcctgcctaatgaacctcactggaacaatccctgtgccttatagaggtaaatgtcttattaggtttccagcatagatgcattttgaatacataataaatatgttgccaaagagttataaccaaattaaacctactttctcagggctttgatgctgatatagattttaacttctattcaaattgaagttcatttggcagtaacacctaacattttcagctttcttaaaacttccttaccaagatatatgaacaaataagtgcctaagttcaccagaggagtttaatgtttcactgaatcaaataattgtagaactagaaatggatcttacttgcctcctagttcagcctcccacaccattcaaactttcctgacaatgggaagctaagatataggcttcaaagtcaggacagagttatgtttgagttcttgctctataatcttagcagttttgttacatgttatatatggatatctttgacatttggataatagtacctaacttgggggaagtgagcatttattagataatgaatgtaaaggacgtggcacagagcctggaatataataagcagacagttaaaagtagctgttatttatggttgtgatgttggtgataatgctaatgatagataattgatctctgttagcctgtctttcaccctctgctgaaatatttagtggtgaggaagcatttagcttaatgagagaccattttgtttttggacagctctgtttgttaattattccttgtaattagctaagacctatgtcctttacccattgatttcacatagtacctgctattcataaggcaataaggataggagctgtggctacaaagatgaataaaggatgtacctcttctggaagaactccactaataggggaaacaaagggaacagatagatatttacactattctgtaactgctatagaattataaaatgaaaaagtgctatgagattacagaggagaacaaggccacatgggaagatcacagaggaagtgacatttgagccactattaataagtcaaccattcataataaacagagggaaaaaacagttaattgagtatcagcattgtataaagcaagtataagtttagggaagactgagtaaaatttaagattactgactttgctattgccctcaggaaataaattctgctgggggaaataggaatgggggggatgcggatatatgtataaattgtatctagactaaacctctgttcctttaaccattcttcaatttaaaaataataaaaatagagtaactgaagtttctatttctttttcaggtaatacatacaatattccaatatgcctatggctactggacacatacccatataatccccctatctgttttgttaagcctactagttcaatgactattaaaacaggaaagcatgttgatgcaaatgggaagatatatcttccttatctacatgaatggaaacacgtaagtattcatagtgttctgtgaattagttatgttttatatattttgctcactagcatctgctttcttttagcactcaaggaggattcgaggtaggatagataa

(19) United States
(12) Patent Application Publication
(10) Pub. No.: US 2005/0118648 A1
(43) Pub. Date: Jun. 2, 2005

(54) Methods and combinations for gene targeting by homologous recombination

(76) Inventor: Limin Li, Potomac, MD (US)

Correspondence Address: Piper Rudnick;Supervisor Patent Prosecution Service, 1200 Nineteenth Street NW, Washigton, DC, 20036-2412, US

(21) Appl. No.: 10/490,830

(22) PCT Filed: Sep. 27, 2002
(86) PCT No.: PCT/US02/31018

(30) Foreign Application Priority Data
Sep. 27, 2001 (US) 60325450

(51) Int. Cl. C12Q 1/68; G01N 33/53; G01N 33/567; C12N 15/85

(52) U.S. Cl. 435/7.2; 435/455; 435/6; 435/325

(57) ABSTRACT

The invention provides methods and compositions for inserting a DNA sequence in the genome of a cell by homologous recombination. In particular, the method utilizes a selection scheme in which a selection marker gene that encodes a fluorescence protein, such as a green fluorescence protein, is used for selection against random, non homologous insertions.

This application claims the benefit under 35 U.S.C. § 119(e) of U.S. Provisional Patent Application No. 60/325,450, filed on Sep. 27, 2001, which is incorporated by reference herein in its entirety.

### 1. FIELD OF THE INVENTION

The invention relates to methods and compositions for gene targeting by homologous recombination. The invention also relates to DNA constructs that can be used for gene targeting by homologous recombination.

### 2. BACKGROUND OF THE INVENTION

Understanding the biological function of mammalian genes remains one of the major challenges in the post genomic era. With the human genome sequenced, less than 20% of the estimated 30,000-50,000 genes (Venter et al, 2001 Science 291:5507; Lander, 2001, Nature 409:860) are well characterized with their biological function known. Gene targeting by homologous recombination is widely used for introducing insertions at targeted genomic loci.

A major problem in gene targeting by homologous recombination is the identification and isolation of cells that have undergone homologous recombination from among a large pool of cells that have undergone random, non-homologous recombination. To circumvent this problem, a method utilizing a positive-negative selection scheme for homologous recombination has been disclosed (see, e.g., U.S. Pat. Nos. 5,487,992; 5,627,059; 5,631,153; and 6,204,061). The method makes use of a vector comprising four DNA sequences: a first DNA sequence which contains at least one sequence portion which is substantially homologous to a portion of a first region of a target DNA sequence; a second DNA sequence containing at least one sequence portion which is substantially homologous to another portion of a second region of a target DNA sequence; a third DNA sequence which is positioned between the first and second DNA sequences and encodes a positive selection marker which when expressed is functional in the target cell in which the vector is used; and a fourth DNA sequence encoding a negative selection marker, also functional in the target cell, which is positioned 5′ to the first or 3′ to the second DNA sequence and is substantially incapable of homologous recombination with the target DNA sequence. In this method, transfection of the cells with the vector produces two different types of cells, one containing random integration of the vector into the genome of the cell and the other containing integration of the vector at the target genomic locus by homologous recombination. Random integration leads to the insertion of all four sequences into the genome, whereas homologous recombination leads to the insertion of only the first through third sequences into the genome. Cells containing integration of the first through third sequences by homologous recombination are selected both positively by way of the positive selection marker and negatively by way of the negative selection marker. However, selection by way of a negative selection marker relies on the use of a selection agent that is toxic to the cells. Such selection may not always be available for all types of cells. Secondly, the method requires culturing the cells under both the positive and negative selection conditions, and therefore, is time consuming. Furthermore, host cells may contain their own genes that encode the negative selection marker, which may cause background problem.

U.S. Pat. No. 5,527,674 discloses a method for homologous recombination using a DNA construct comprising a positive selection marker and a negative selection system “antagonistic” to the expression of the positive selection marker. The negative selection system is situated outside the homologous regions and comprises an antisense gene which, when expressed, prevents the expression of the positive selection marker. Cells that have undergone homologous recombination can therefore be selected solely based on the presence of the positive selection marker activity. However, the method relies on, among others, a DNA construct design in which the promoter for the positive selection marker must be weaker than the promoter for the antisense gene for effective inhibition of the positive selection marker. This requirement of using a weak promoter for the positive selection marker significantly limits the choice of promoters that can be used for efficient selection.

U.S. Pat. No. 6,284,541 discloses a method for homologous recombination. The method utilizes a cell surface marker for selection against random integrations. Selection for the absence of the negative selection marker is carried out by contacting the transfected cells with a binding molecule, e.g., a fluorescence-dye-tagged antibody, and identifying and isolating the cells using, e.g., a fluorescence activated cell sorter (FACS). Since the method relies on binding of a binding molecule to the selection marker expressed on the surface of the transfected cells, background due to non-specific binding may be significant. It is also known that the sensitivity and resolution of a method based on staining using a fluorescence dye-labeled antibody can be low (see, e.g., Wang et al., 1994, Nature 639:400-403). Further, although this method does not require the use of a toxic agent for negative selection, it still involves a separate step of contacting the transfected cells with one or more gents, e.g., a primary antibody and a fluorescence dye-labeled secondary antibody, therefore incurring further time and cost.

More efficient methods for gene targeting by homologous recombination are desirable for large scale gene knockout and function analysis. There is therefore a need for methods that allow more efficient identification and isolation of cells that have undergone homologous recombination from a large pool of cells that have undergone random, non-homologous recombination. In particular, there is a need for methods that have minimum background problem and require fewer rounds of separate steps.

Discussion or citation of a reference herein shall not be construed as an admission that such reference is prior art to the present invention.

### 3. SUMMARY OF THE INVENTION

The invention relates to methods and compositions for inserting a DNA sequence in the genome of cells of a cell type by homologous recombination. The method of the invention utilizes a gene targeting vector comprising a sequence region that encodes a fluorescence protein, such as but not limited to a green fluorescence protein, located outside the homologous sequence regions for selection against random, non-homologous insertions.

The invention provides gene targeting vectors comprising sequences encoding a positive selection marker for selection for integration of all or portion of the gene targeting vector in the genome of the target cells and at least one fluorescence marker for selection against random integration of the vector in the genome of the target cells. The gene targeting vector of the invention comprises four sequence regions: a first sequence region comprising a nucleotide sequence which is substantially homologous to a first target DNA sequence in the target genome; a second sequence region comprising a nucleotide sequence which is substantially homologous to a second target DNA sequence in the target genome; a third sequence region positioned between the first and second DNA sequence regions and comprising a nucleotide sequence that encodes a positive selection marker; and a fourth sequence region comprising a nucleotide sequence located at 5′ to the first or 3′ to the second sequence region encoding a fluorescence marker for selection against random integration.

The positive selection marker gene can be any gene encoding a measurable and selectable marker in the type of cells, e.g., a type of mammalian cells, known in the art, including but not limited to, a drug resistance gene, such as but not limited to Neomycin/G418, Puromycin, Hygromycin B, Zeocin, or mycophenolic acid resistance gene; a gene encoding a cell surface marker, such as but not limited to a gene encoding CD4, CD8, CD20, HA, or any synthetic or foreign cell surface marker; a gene encoding a fluorescent marker, such as but not limited to a gene encoding green fluorescence protein (GFP), blue fluorescence protein (BFP), red fluorescence protein (RFP), or any variants thereof; a gene encoding β-galactosidase; and a gene is a gene encoding β-geo. The positive selection marker gene can also encode a combination of more than one positive selection marker, such as but not limited to a gene that encodes a rsGFP-neo fusion protein.

The third sequence region can also comprise regulatory sequences regulating the expression of the positive selection marker. In one embodiment, the third sequence region comprises a regulatory sequence comprising a promoter, either regulated or constitutive, that regulates the expression of the positive selection marker gene. The regulatory sequences can also comprise other sequences that facilitate expression of the positive selection marker, e.g., enhancers.

The third sequence region can further comprise any other sequences to be inserted into the genome of the target cells. In one embodiment, the third sequence region comprises a regulated expression sequence portion comprising a regulated promoter and a selection marker under the control of the regulated promoter. The regulated promoter can be any transcription regulation system known in the art for the type of cells chosen, including but not limited to a tetracycline regulated gene expression system.

In embodiments in which a regulated expression sequence portion is included, the selection marker gene in the regulated expression sequence portion can be any selection marker that can be expressed in the chosen type of cells, e.g., a chosen type of mammalian cells, known in the art, including but not limited to, drug resistance genes, such as but not limited to Neomycin/G418, Puromycin, Hygromycin B, Zeocin, or mycophenolic acid resistance genes; cell surface marker genes, such as but not limited to genes encoding CD4, CD8, CD20, HA, or any synthetic or foreign cell surface markers; genes encoding fluorescence markers, such as but not limited to genes encoding green fluorescence protein (GFP), blue fluorescence protein (BFP), red fluorescence protein (RFP), or any variants thereof. The selection marker expressed by the selection marker gene in the regulated expression portion can be the same as or different from the positive selection marker. In a preferred embodiment, the selection marker expressed by the selection marker gene in the regulated expression portion is different from the positive selection marker.

The third sequence region of the gene targeting vector can still further comprise an optional rapid cloning element comprising a bacterial plasmid replication origin and a bacterial selection marker. Preferably, the replication origin sequence comprises all necessary sequences for initiation of replication and segregation. Any bacterial plasmid replication origin, such as but not limited to Ori, colEI, pSC101, pUC, or f1 phage ori, can be used. Any bacterial selection markers, such as but not limited to, chloramphenicol, ampicillin, tetracycline, or kanamycin can be used in the present invention.

The fourth sequence region comprises a selection marker gene encoding a fluorescence marker, e.g., a green fluorescence marker to permit fluorescence based selection against random integration of the gene targeting vector in the genome of the target cells. The fourth sequence region is located outside the homologous sequence regions, i.e., at 5′ to the first or 3′ to the second sequence region. Fluorescent markers that can be used in the present invention include, but are not limited to, genes encoding green fluorescence protein (GFP), blue fluorescence protein (BFP), red fluorescence protein (RFP), or any variants thereof. When a fluorescence marker is used as the positive selection marker, it is preferable that the selection marker encoded in the fourth sequence region is a fluorescence marker that has distinguishable excitation and/or emission characteristics from the positive selection marker. In a preferred embodiment, the positive selection marker and the selection marker encoded in the fourth sequence region are one or the other combination of rsGFP and BFP from Qbiogene (Carlsbad, Calif.).

The gene targeting vector can further comprise an optional fifth sequence region comprising a nucleotide sequence encoding a selection marker for selection against random integration, which is located at the opposite end of the gene targeting vector from the fourth sequence region, i.e., at 5′ to the first if the fourth sequence region is located at the 3′ to the second sequence region, or at 3′ to the second sequence region if the fourth sequence region is located at the 5′ to the first sequence region. The selection marker encoded in the fifth sequence region can be a negative selection marker. Alternatively, the selection marker encoded in the fifth sequence region can be any one of the fluorescence markers. In embodiments in which the selection marker encoded in the fifth sequence region is a fluorescence marker, it can be the same as or different from the fluorescence marker encoded in the fourth sequence region. When a fluorescence marker is used as the positive selection marker, it is preferable that the selection marker encoded in the fifth sequence region is a fluorescence marker that has distinguishable excitation and/or emission characteristics from the positive selection marker.

The invention provides methods for generating a plurality of cells comprising cells that carry an insertion of a DNA sequence in the genome by homologous recombination. The method of the invention comprises transfecting cells of a chosen cell type with a gene argeting vector of the invention, e.g., a gene targeting vector comprising: a first sequence region comprising a nucleotide sequence which is substantially homologous to a first target NA sequence in the genome of cells of the chosen cell type; a second sequence region comprising a nucleotide sequence which is substantially homologous to a second target DNA sequence in the genome of cells of the chosen cell type; a third sequence region located between said first and second sequence regions, comprising a nucleotide sequence that encodes a positive selection marker; and a fourth sequence region comprising a nucleotide sequence encoding a fluorescence marker, located at 5′ to said first or 3′ to said second sequence region, wherein said positive selection marker is expressed in said cells that carry said insertion by homologous recombination, and wherein said fluorescence marker encoded in said fourth sequence region is not expressed in said cells that carry said insertion by homologous recombination.

In the methods of the invention, the plurality of cells comprising cells that carry an insertion of a DNA sequence in the genome by homologous recombination can be selected by selecting for the presence of the positive selection marker activity and the absence of the activity of the selection marker or markers encoded in those outside regions, i.e., the fourth and/or the fifth sequence regions. In a preferred embodiment, a drug resistance gene is used as the positive selection marker. In this embodiment, the selection for cells carrying the insertion of the positive selection marker gene can be achieved by culturing the transfected cells in the presence of the corresponding drug. In another preferred embodiment, a fluorescence marker is used as the positive selection marker. In this embodiment, the selection for cells carrying the insertion of the positive selection marker gene can be achieved by any fluorescence based cell sorting methods known in the art, e.g., by FACS. The selection against random, non-homologous, integration of the gene targeting vector can be carried out by detecting the fluorescence from the fluorescence marker encoded in the fourth sequence region using any fluorescence based cell sorting methods known in the art, e.g., by FACS. The step of selection against random, non-homologous, integration of the gene targeting vector can be carried out before, concurrently with, or after the step of selection for the presence of the positive selection marker. When a fluorescence based cell sorting method is used for selection for the presence of the positive selection marker and/or against the presence of the fluorescence markers encoded in the outside regions, the fluorescence window is preferably set such that the cells that carry the insertion of the DNA sequence by homologous recombination constitute at least 10%, 30%, 50%, 70%, or 90% of the plurality of cells.

Cells that are selected can be further characterized by any methods known in the art. In one embodiment, standard PCR and sequencing procedures are used to characterize the cells. In another embodiment, cells are characterized by making use of the rapid cloning element. In this embodiment, genomic regions carrying the insertions are characterized by restriction digesting the rapid cloning element and its flanking genomic DNA, recirculizing by DNA ligation, and transfecting into bacterial cells. The plasmids isolated from transformed bacteria are used to determine DNA sequence of the flanking genomic sequences by any DNA sequencing methods known in the art.

### 4. BRIEF DESCRIPTION OF FIGURES

FIG. 1 shows a schematic illustration of the method of the invention.

FIG. 2 shows exemplary configurations of gene targeting vectors of the invention.

FIG. 3 shows the restriction map of gene targeting vector 1.

FIG. 4 shows the restriction map of gene targeting vector 2.

FIG. 5 shows the restriction map of gene targeting vector 3.

FIGS. 6A and B show sequences of homologous recombination region 1 (SEQ ID NO:1) and homologous recombination region 2 (SEQ ID NO:2) for targeting the human TSG 101 gene.

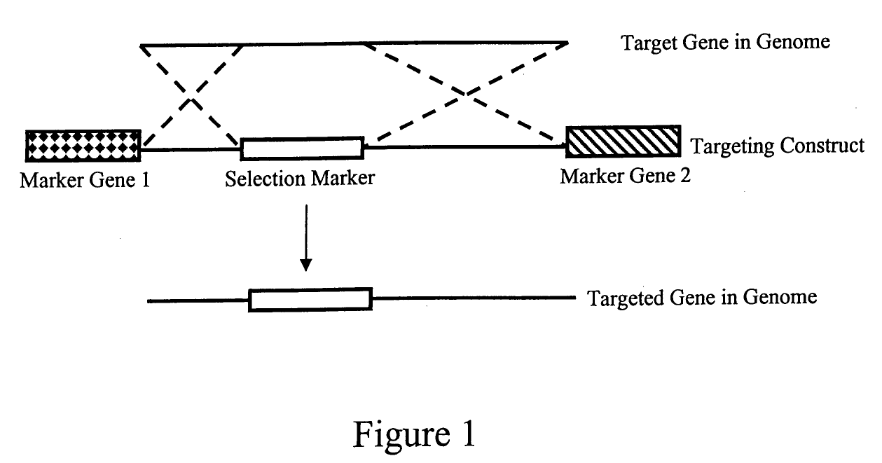

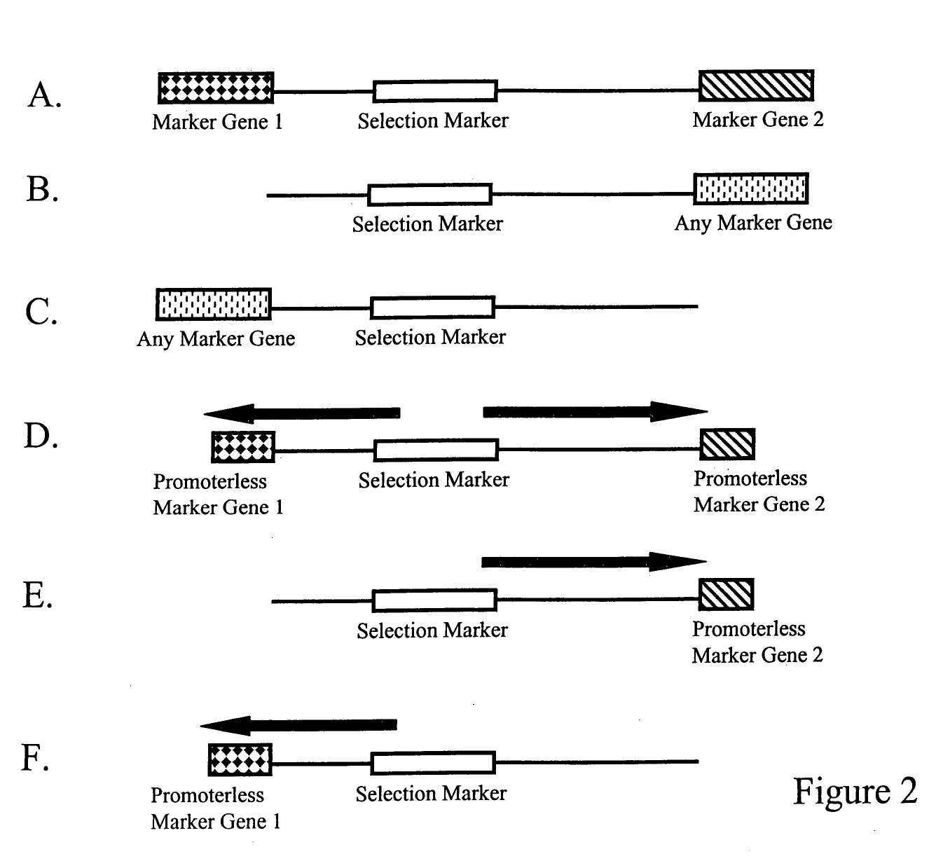

### 5. DETAILED DESCRIPTION OF THE INVENTION

The invention provides methods and compositions for inserting a DNA sequence in the genome of cells of a cell type by homologous recombination. The method of the invention utilizes a gene targeting vector comprising a sequence region that encodes a fluorescence protein, such as but not limited to a green fluorescence protein, located outside the homologous sequence regions, for selection against random, non-homologous insertions.

The method of the invention can be used to target any genomic sequences in any cells, including but not limited to, any plant or animal cells, e.g., mammalian cells. Any cell type can be used in the present invention, including but not limited to, somatic cells and stem cells.

### 5.1. Gene Targeting Vectors

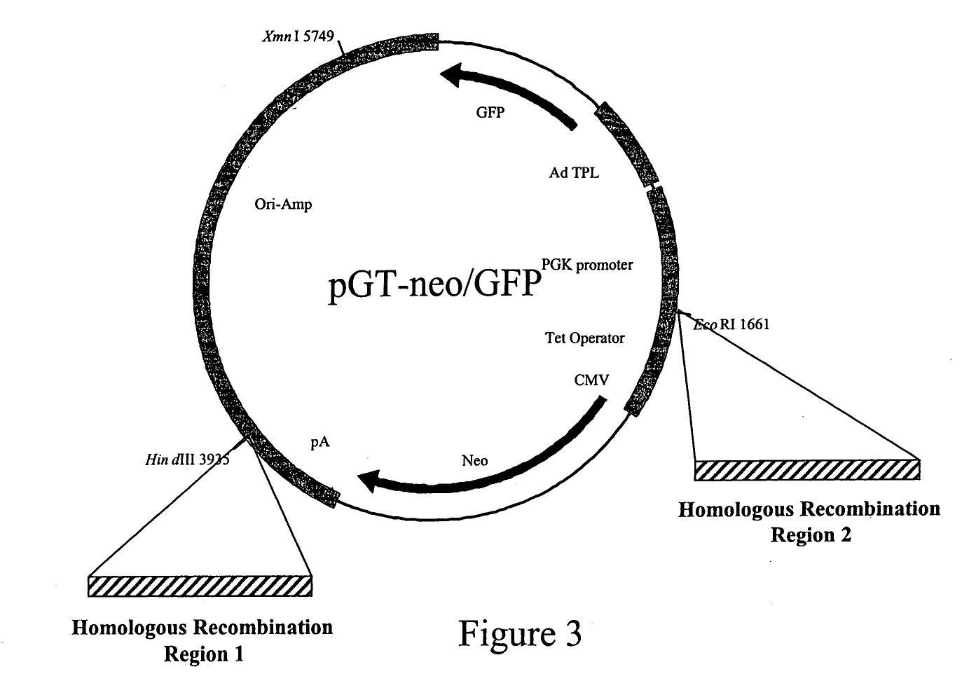

The invention provides gene targeting vectors comprising sequences encoding a positive selection marker for selection for integration of all or portion of the gene targeting vector in the genome of the target cells and at least one fluorescence marker for selection against random integration of the vector in the genome of the target cells. The gene targeting vector of the invention comprises four sequence regions: a first sequence region comprising a nucleotide sequence which is substantially homologous to a first target DNA sequence in the target genome; a second sequence region comprising a nucleotide sequence which is substantially homologous to a second target DNA sequence in the target genome; a third sequence region positioned between the first and second DNA sequence regions and comprising a nucleotide sequence that encodes a positive selection marker; and a fourth sequence region comprising a nucleotide sequence located at 5′ to the first or 3′ to the second sequence region encoding a fluorescence marker for selection against random integration. (See, e.g., FIGS. 3-5 for exemplary gene targeting vectors) The DNA construct can further comprise an optional fifth sequence region comprising a nucleotide sequence encoding a selection marker for selection against random integration, which fifth sequence region is located at the opposite end of the gene targeting vector from the fourth sequence region, i.e., at 5′ to the first if the fourth sequence region is located at the 3′ to the second sequence region, or at 3′ to the second sequence region if the fourth sequence region is located at the 5′ to the first sequence region. When a cell is transfected with the gene targeting vector of the invention, homologous recombination at the targeted genomic locus results in the integration of the first through third sequence regions at the targeted locus and the loss of the selection marker gene or genes located in the fourth and the fifth, if applicable, sequence regions. Cells carrying an insertion at the targeted locus can therefore be identified by the presence of the activity of the positive selection marker encoded by the third sequence region and the absence of fluorescence of the fluorescence protein or proteins encoded by the fourth and/or fifth sequence regions.

Each of the first and second sequence regions comprises a nucleotide sequence that is substantially homologous to a sequence at the target genomic locus. As used herein, “substantially homologous” refers to a degree of homology between the two DNA sequences that is at least 25%. Preferably, each of the homologous sequences is at least 20 bp, more preferably at least 200 bp, still more preferably at least 1 kbp, and most preferably at least 2.5 kbp in length. The degree of homology between each of the homologous sequences and the corresponding target sequence is preferably at least 50%, more preferably t least 75%, still more preferably at least 90%, and most preferably 100%. Once a target sequence region in the genome of a target cell is given, one skilled in the art will be able to select homologous sequences that can be used in targeting the sequence region.

The third sequence region comprises a nucleotide sequence that encodes a positive selection marker. The positive selection marker gene can be any gene encoding a measurable and selectable marker in the type of cells, e.g., a type of mammalian cells, known in the art. In one embodiment, the positive selection marker gene is a gene encoding β-galactosidase. In another embodiment, the positive selection marker gene is a gene encoding β-geo. In still another embodiment, the positive selection marker gene is a drug resistance gene, such as but not limited to Neomycin/G418, Puromycin, Hygromycin B, Zeocin, or mycophenolic acid resistance gene. In still another embodiment, the positive selection marker gene is a gene encoding a cell surface marker, such as but not limited to a gene encoding CD4, CD8, CD20, HA, or any synthetic or foreign cell surface marker. The positive selection marker gene can also be a gene encoding a fluorescent marker, such as but not limited to a gene encoding green fluorescence protein (GFP), blue fluorescence protein (BFP), red fluorescence protein (RFP), or any variants thereof (see, e.g., Autofluorescent Proteins available at http://www.qbiogene.com/protocols/gene-expression/m-afp.pdf (accessed Sep. 5, 2001); Ellenberg et al., 1999, Trends in Cell Biol 9:52-56; Mizuno et al., 2001, Biochem. 40:2502-10; and Living Colors® User Manual, published Aug. 30, 2000, available at http://www.clontech.com/techinfo/manuals/PDF/PT2040-1.pdf (accessed Sep. 5, 2001)). In a preferred embodiment, the positive selection marker gene comprises a splicing acceptor at its 5′ end that allows fusion of the positive selection marker gene to the RNA transcript from the upstream exons (see, e.g., Li et al., 1996, Cell 85:319-329). The positive selection marker gene can also encode a combination of more than one positive selection marker. In one embodiment, the positive selection marker gene encodes a rsGFP-neo fusion protein (see, e.g., Autofluorescent Proteins available at http://www.qbiogene.com/protocols/gene-expression/m-afp.pdf). It will be apparent to one skilled in the art that any positive selection marker genes that are functionally equivalent to any of the positive selection marker gene as described, including any genes that are modified or mutated from any of the described positive selection marker genes, are also within the scope of the present invention.

The third sequence region can also comprise regulatory sequences regulating the expression of the positive selection marker. In one embodiment, the third sequence region comprises a regulatory sequence comprising a promoter that regulates the expression of the positive selection marker gene. This is especially useful when the DNA construct is inserted at a genomic locus to activate an inactive endogenous gene. The regulatory sequences can also comprise other sequences that facilitate expression of the positive selection marker, e.g., enhancers. Any regulatory sequences, e.g., regulated or constitutive promoters, enhancers, etc., known in the art can be used. One skilled in the art will be able to choose the appropriate regulatory sequences for this purpose.

The third sequence region can also comprise any other sequences to be inserted into the genome of the target cells (see, e.g., Limin Li, U.S. Provisional Patent Application No. 60/325,497, filed on Sep. 27, 2001, which is incorporated herein by reference in its entirety). In one embodiment, the third sequence region comprises a regulated expression sequence portion comprising a regulated promoter and a selection marker under the control of the regulated promoter. The regulated promoter can be any transcription regulation system known in the art that can be used in the chosen type of cells (see, e.g., Gossen et al, 1995, Science 268:1766-1769; Lucas et al, 1992, Annu. Rev. Biochem. 61:1131; Li et al., 1996, Cell 85:319-329; Saez et al., 2000, Proc. Natl. Acad. Sci. USA 97:14512-14517; and Pollock et al., 2000, Proc. Natl. Acad. Sci. USA 97:13221-13226). In one embodiment, a tetracycline regulated gene expression system is used (see, e.g., Gossen et al, 1995, Science 268:1766-1769). In another embodiment, an ecdysone regulated gene expression system is used (see, e.g., Saez et al., 2000, Proc. Natl. Acad. Sci. USA 97:14512-14517). In still another embodiment, a MMTV glucocorticoid response element regulated gene expression system is used (see, e.g., Lucas et al, 1992, Annu. Rev. Biochem. 61:1131). Other protein or chemical regulated gene expression systems can also be used (see, e.g., Li et al., 1996, Cell 85:319-329).

The selection marker gene in the regulated expression sequence portion can be any selection marker that can be expressed in the chosen type of cells, e.g., a chosen type of mammalian cells, known in the art. In one embodiment, a drug resistance gene is used as the selection marker. Drug resistance genes that can be used in the present invention include, but are not limited to, Neomycin/G418, Puromycin, Hygromycin B, Zeocin, or mycophenolic acid resistance genes. In another embodiment, a cell surface marker is used as the selection marker. Cell surface marker genes that can be used in the present invention include, but are not limited to, genes encoding CD4, CD8, CD20, HA, or any synthetic or foreign cell surface markers. In still another embodiment, a fluorescence marker is used as the selection marker. Fluorescent markers that can be used in the present invention include, but are not limited to, genes encoding green fluorescence protein (GFP), blue fluorescence protein (BFP), red fluorescence protein (RFP), or any variants thereof (see, e.g., Autofluorescent Proteins available at http://www.qbiogene.com/protocols/gene-expression/m-afp.pdf (accessed Sep. 5, 2001); Ellenberg et al., 1999, Trends in Cell Biol 9:52-56; Mizuno et al., 2001, Biochem. 40:2502-10; and Living Colors® User Manual, published Aug. 30, 2000, available at http://www.clontech.com/techinfo/manuals/PDF/PT2040-1.pdf (accessed Sep. 5, 2001)). The selection marker expressed by the selection marker gene in the regulated expression portion can be the same as or different from the positive selection marker. In a preferred embodiment, the selection marker gene expressed by the selection marker gene in the regulated expression portion is different from the positive selection marker.

In embodiments where a regulated expression sequence portion is included, the regulated expression sequence portion can be placed in either orientation in relation to other components in the gene targeting vector. In a preferred embodiment, the regulated expression sequence portion is oriented in the opposite orientation as the positive selection marker. In such an embodiment, the regulated expression sequence portion can be located either upstream or downstream of the positive selection marker gene. In another embodiment, in which a regulatory sequence is included to activate the expression of the positive selection marker gene, the regulated expression sequence portion is oriented in the same orientation as the positive selection marker gene.

The third sequence region of the gene targeting vector can also comprise an optional rapid cloning element comprising a bacterial plasmid replication origin and a bacterial selection marker. As used herein, a “rapid cloning element” refers to a nucleotide sequence which can be used to facilitate the cloning of the genomic sequences flanking the integration site in a host, e.g., in a bacterial host. In the present invention, a rapid cloning element comprising a replication origin is often used. As used herein, an “origin” or “replication origin” refers to a bacterial replication origin sequence. Preferably, the replication origin sequence comprises all necessary sequences for initiation of replication and segregation. Any bacterial plasmid replication origin, such as but not limited to Ori, colEI, pSC101, pUC, or f1 phage ori can be used. Any bacterial selection markers, such as but not limited to, chloramphenicol, ampicillin, tetracycline, or kanamycin can be used in the present invention. The rapid cloning element functions as a selection bacterial plasmid to allow efficient cloning of the genomic DNA sequences flanking it into bacterial cells.

The fourth sequence region comprises a selection marker gene encoding a fluorescence marker, e.g., a green fluorescence marker. The fourth sequence region is located outside the homologous sequence regions, i.e., at 5′ to the first or 3′ to the second sequence region. Fluorescent markers that can be used in the present invention include, but re not limited to, genes encoding green fluorescence protein (GFP), blue fluorescence protein (BFP), red fluorescence protein (RFP), or any variants thereof (see, e.g., Autofluorescent Proteins available at http://www.qbiogene.com/protocols/gene-expression/m-afp.pdf (accessed Sep. 5, 2001); Ellenberg et al., 1999, Trends in Cell Biol 9:52-56; Mizuno et al., 2001, Biochem. 40:2502-10; and Living Colors® User Manual, published Aug. 30, 2000, available at http://www.clontech.com/techinfo/manuals/PDF/PT2040-1.pdf (accessed Sep. 5, 2001)). When a fluorescence marker is used as the positive selection marker, it is preferable that the selection marker encoded in the fourth sequence region is a fluorescence marker that has distinguishable excitation and/or emission characteristics from the positive selection marker. In a preferred embodiment, the positive selection marker and the selection marker encoded in the fourth sequence region are one or the other combination of rsGFP and BFP from Qbiogene (Carlsbad, Calif.).

The gene targeting vector can optionally comprise a fifth sequence region comprising a selection marker gene for selection against random, non-homologous, recombination. The selection marker encoded by the selection marker gene in the fifth sequence region can be a negative selection marker. Any negative selection marker known in the art can be used in the invention, including but not limited to HSV-tk, Hprt, and Gpt. The selection marker encoded by the selection marker gene in the fifth sequence region can also be a fluorescence marker, which is different from the fluorescence marker used as the positive selection marker, if a fluorescence marker is used as the positive selection marker. The fluorescence marker encoded by the fifth sequence region can be the same as or different from the fluorescence marker encoded in the fourth sequence region. In one embodiment, the fluorescence marker encoded by the fifth sequence region is the same as the fluorescence marker encoded in the fourth sequence region. In this embodiment, the population of cells containing at least one of the fluorescence markers in their genomes is selected by detecting the fluorescence marker. In another embodiment, the fluorescence marker encoded by the fifth sequence region is different from the fluorescence marker encoded in the fourth sequence region. In a preferred embodiment, the fluorescence marker encoded by the fifth sequence region has distinguishably different emission and/or excitation wavelengths as compared to the fluorescence marker encoded in the fourth sequence region. In this embodiment, the populations of cells containing different fluorescence markers in their genomes can be selected and separated by detecting the different fluorescence markers. Fluorescent markers that can be used in the present invention include, but are not limited to, genes encoding green fluorescence protein (GFP), blue fluorescence protein (BFP), red fluorescence protein (RFP), or any variants thereof (see, e.g., Autofluorescent Proteins available at http://www.qbiogene.com/protocols/gene-expression/m-afp.pdf (accessed Sep. 5, 2001); Ellenberg et al., 1999, Trends in Cell Biol 9:52-56; Mizuno et al., 2001, Biochem. 40:2502-10; and Living Colors® User Manual, published Aug. 30, 2000, available at http://www.clontech.com/techinfo/manuals/PDF/PT2040-1.pdf (accessed Sep. 5, 2001)). The fifth sequence region is located at the opposite end of the gene targeting vector from the fourth sequence region, i.e., at 5′ to the first if the fourth sequence region is located at the 3′ to the second sequence region, or at 3′ to the second sequence region if the fourth sequence region is located at the 5′ to the first sequence region. The inclusion of the fifth sequence region comprising another selection marker for selection against random integration is useful in enhancing selection against random insertions in which all or part of the selection marker encoded in the fourth sequence region is excised before random insertion occurs.

Depending on the particular gene targeting vector used, additional sequences may be necessary for inclusion in the vector. For example, the gene targeting vector may contain restriction sites to facilitate the manipulation of the vector. The gene targeting vector may also contain sequences that aid the integration of the vector into the host genome. Such sequences and the manner of their inclusion in the vector are well within the knowledge of anyone skilled in the art and will be apparent to anyone skilled in the art when a particular vector is chosen.

### 5.2. Methods for Identification and Isolation of Cells

The gene targeting vectors can be introduced into mammalian cells by any DNA transfection methods known in the art, such as microinjection, electroporation and LIPOFECTAMINE.

The transfection of the cells using the gene targeting vector can result in two types of insertion events: insertion by homologous recombination at the target genomic locus and random insertion of the gene targeting vector in the genome. Insertion by homologous recombination at the target locus leads to the integration of the nucleotide sequence between the first and second sequence regions, i.e., the homologous sequences, into the target genome and the excision of any sequence(s) outside the homologous sequence regions, i.e., 5′ of the first sequence region and 3′ of the second sequence region. Therefore, cells that have undergone homologous recombination can be identified by the presence of the positive selection marker activity and the absence of the activity of the selection marker or markers encoded in those outside regions, i.e., the fourth and/or the fifth sequence regions. Random insertion of the gene targeting vector in the host genome, on the other hand, leads to the integration of the entire vector into the genome. Cells that have undergone random insertion can therefore be identified by the presence of both the positive selection marker and the activity of the selection marker or markers encoded in those outside regions. The gene targeting vector of the invention can be integrated into the genome of transfected cells in two configurations. In one embodiment, the gene targeting vector integrates behind a chromosomal promoter. In this embodiment, the positive selection marker gene is turned on by the chromosomal promoter. Integration of the gene targeting vector results in disruption of transcription at the allele. In another embodiment, the gene targeting vector integrates upstream of an inactive or active chromosomal promoter. In this embodiment, integration of the gene targeting vector activates the inactive chromosomal promoter or amplify the active chromosomal promoter. This embodiment allows activation of chromosomal genes in cells to screen for any phenotypic changes associated to the activated gene.

The selection for the presence of the positive selection marker can be carried out by standard methods known in the art, depending on the positive selection marker used. For example, in one preferred embodiment, a drug resistance gene is used as the positive selection marker. In this embodiment, the selection for cells carrying the insertion of the positive selection marker gene can be achieved by culturing the transfected cells in the presence of the corresponding drug. The optimal conditions for selection for insertion of the positive selection marker gene, e.g., concentration of the drug, duration of culturing, etc., can be determined by one skilled in the art once the particular gene is chosen. In another preferred embodiment, a fluorescence marker is used as the positive selection marker. In this embodiment, the selection for cells carrying the insertion of the positive selection marker gene can be achieved by any fluorescence based cell sorting methods known in the art. For example, the selection can be carried out using a FACS system. Any FACS system can be used in the present invention. Preferably, a FACS system equipped with multiple excitation lasers is used to permit concurrent selection of both the positive selection marker and the fluorescence marker encoded in the fourth sequence region. One skilled in the art will be able to determine the parameters for the FACS scan, e.g., excitation/emission wavelengths, widths of fluorescence windows, etc., once the fluorescence marker is chosen. Preferably, the fluorescence window is set such that at least 10% of the sorted cells from the initial cell population are cells having the positive selection marker integrated in the their genomes. More preferably, the fluorescence window is set such that at least 50% of the sorted cells from the initial cell population are cells having the positive selection marker integrated in the their genomes. Still more preferably, the fluorescence window is set such that at least 70% of the sorted cells from the initial cell population are cells having the positive selection marker integrated in the their genomes. Most preferably, the fluorescence window is set such that at least 90% of the sorted cells from the initial cell population are cells having the positive selection marker integrated in the their genomes.

The selection against random, non-homologous integration of the gene targeting vector can be carried out by selecting cells that do not carry the insertion of the fluorescence marker gene encoded in the fourth sequence region of the gene targeting vector. The selection can be achieved using any fluorescence based cell sorting methods known in the art. The step of selection against random, non-homologous integration of the gene targeting vector can be carried out before, concurrently with, or after the step of selection for the presence of the positive selection marker. Depending on the combination of the positive selection marker and the fluorescence marker encoded by a DNA sequence in the fourth sequence region, it will be apparent to one skilled in the art to determine the optimal sequence of the two steps of selections. In a preferred embodiment, when a drug resistance gene is used as the positive selection marker, the step of selection against random, non-homologous, integration is carried out after the step of selection for the presence of the positive selection marker. In another preferred embodiment, when a gene encoding a fluorescence marker is used as the positive selection marker, the step of selection against random, non-homologous, integration can be carried out concurrently with the step of selection for the presence of the positive selection marker.

In one embodiment, the step of selection against random, non-homologous integration is carried out using a standard FACS system. Any FACS system can be used in the present invention. One skilled in the art will be able to determine the parameters for the FACS machine, e.g., excitation/emission wavelengths, fluorescence windows, etc., once the fluorescence marker is chosen. Preferably, the fluorescence window is set such that at least 10% of the sorted cells from the initial cell population are cells that do not carry the insertion of the fluorescence marker gene encoded in the fourth sequence region of the gene targeting vector. More preferably, the fluorescence window is set such that at least 30%, 50%, 70%, or 90% of the sorted cells from the initial cell population are cells that do not carry the insertion of the fluorescence marker gene encoded in the fourth sequence region of the gene targeting vector.

Cells that are selected can be characterized by standard methods known in the art. In ne embodiment, standard PCR and sequencing procedures are used to characterize the ells.

In another embodiment, cells are characterized by making use of the rapid cloning element. In this embodiment, homozygous mutations are characterized by the following steps: first, the rapid cloning element and its flanking genomic DNA are linerized by a single or two compatible restriction enzymes, then recirculized by DNA ligation, and transfected into bacterium. The plasmids isolated from transformed bacteria are used to determine DNA sequence of the flanking exons by any DNA sequencing methods known in the art.

### 6. EXAMPLES

The following examples are presented by way of illustration of the present invention, and are not intended to limit the present invention in any way. In particular, the examples presented hereinbelow describe insertion of pGT-neo/GFP/BFP and pGT-GFP/BFP in the TSG101 locus of the genome of human fibroblast cell line CLL212 (ATCC). This cell line was either transfected with a pTet-off or pTet-On expression vector (Clontech), clones that have the optional expression of transactivator (either TetR or rTetR) were identified by their ability to transactivate a Tet response vector that expresses a detectable marker beta-galactosidase (Clontech). This modified cell line is designated as CLL212-Trans.

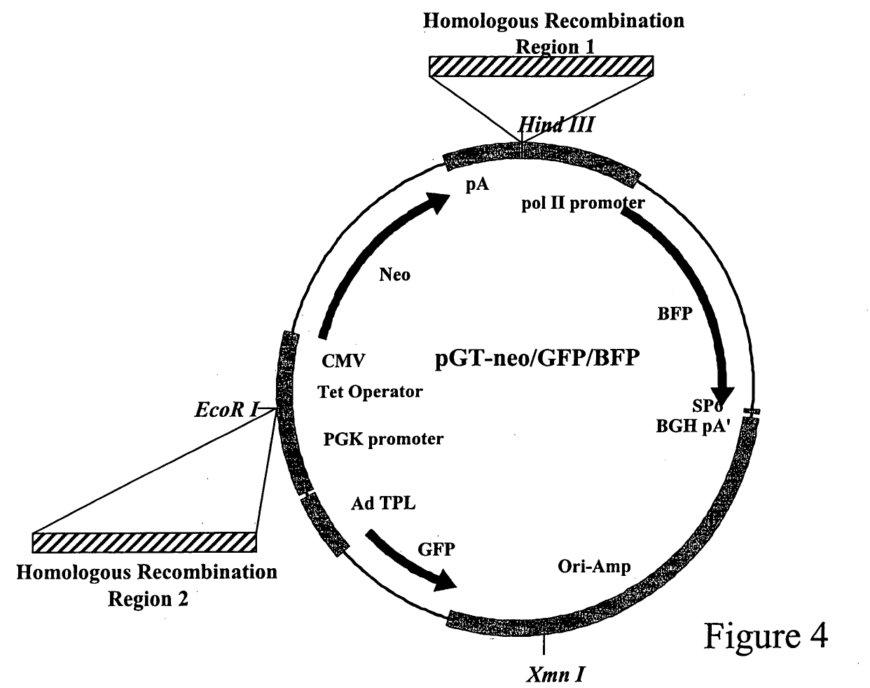

Gene targeting vector depicted in FIG. 4 was constructed as follows: a neo fragment from pSV2neo (Clontech) was inserted into a tetracycline regulated expression vector pUHD 10-3 (http://www.zmbh.uniheidelberg.de/bujard/homepage.html, accessed Sep. 20, 2001) to give pTet-neo. An sgGFP expression cassette and a sgBFP expression cassette were inserted into pTet-neo as shown in FIG. 4 to generate pGT-neo/GFP/BFP.

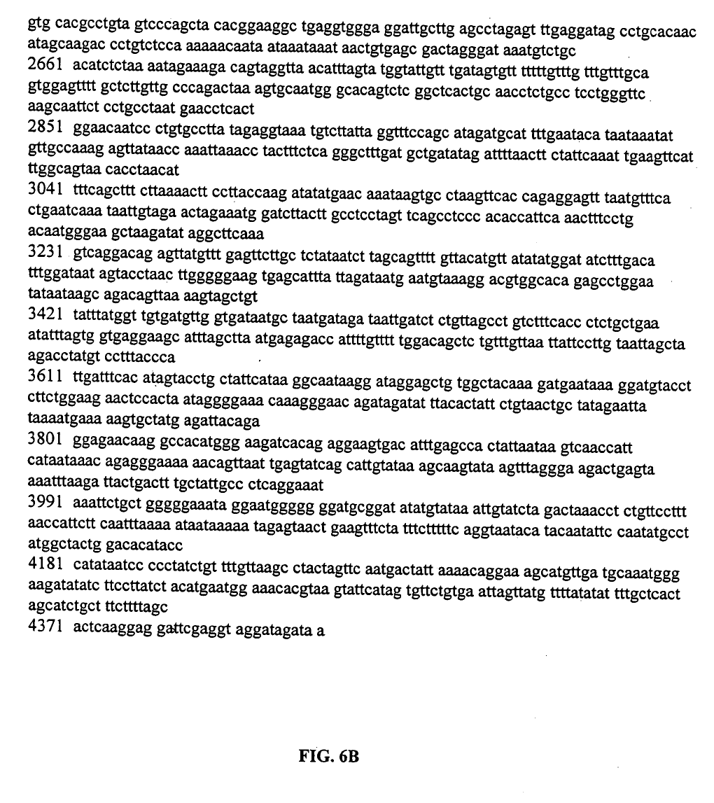

To target the TSG101 locus, a 4 kb region of TSG101 gene that spans exons 4-6 was chosen (GENEBANK® accession no. NT_{—}009307.5). This 4 kb fragment was divided into homologous recombination region 1 (SEQ ID NO:1) and homologous recombination region 2 (SEQ ID NO:2), each region has about 2 kb in length (see FIGS. 6A-B). Homologous recombination region 1 was inserted into pGT-neo/GFP/BFG at a Hind III site, and homologous recombination region 2 was inserted at an EcoR I site to give pGT-neo/GFP/BFP-TSG101. CLL212-trans cells were transfected with the gene targeting vector (pGT-neo/GFP/BFP-TSG101) by electroporation (Li et al., 1996, Cell 85:319-329). Transfected cells are first cultured for 24 to 48 hours and then further cultured in the presence of G418 (400 ug/ml) for 7-10 days. G418 resistance clones were screened under a fluorescence microscope for the expression of GFP and BFP. G418 resistance clones that did not express any of the GFP and BFP were isolated and expanded into cell lines. These clones were confirmed to have undergone the desired homologous recombination at the TSG101 locus by genomic Southern blotting analysis and PCR analysis. Western blotting using a rabbit anti-TSG101 antibody (CLONETECH, see also Li et al., Proc. Natl. Acad. Sci. USA, 98:1619-24) further confirmed the inactivation of TSG101 protein production.

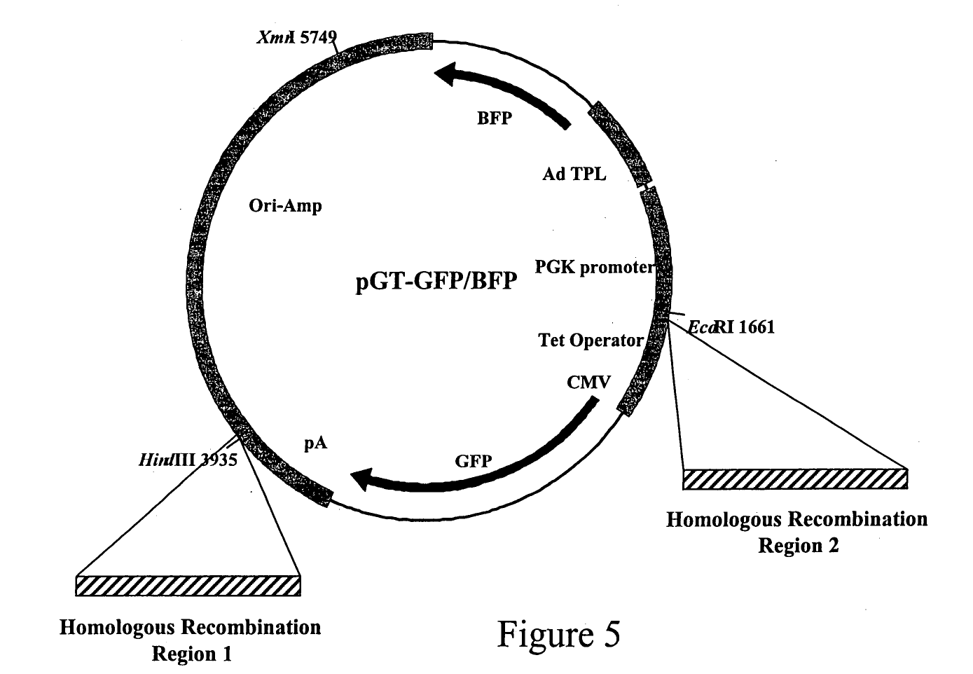

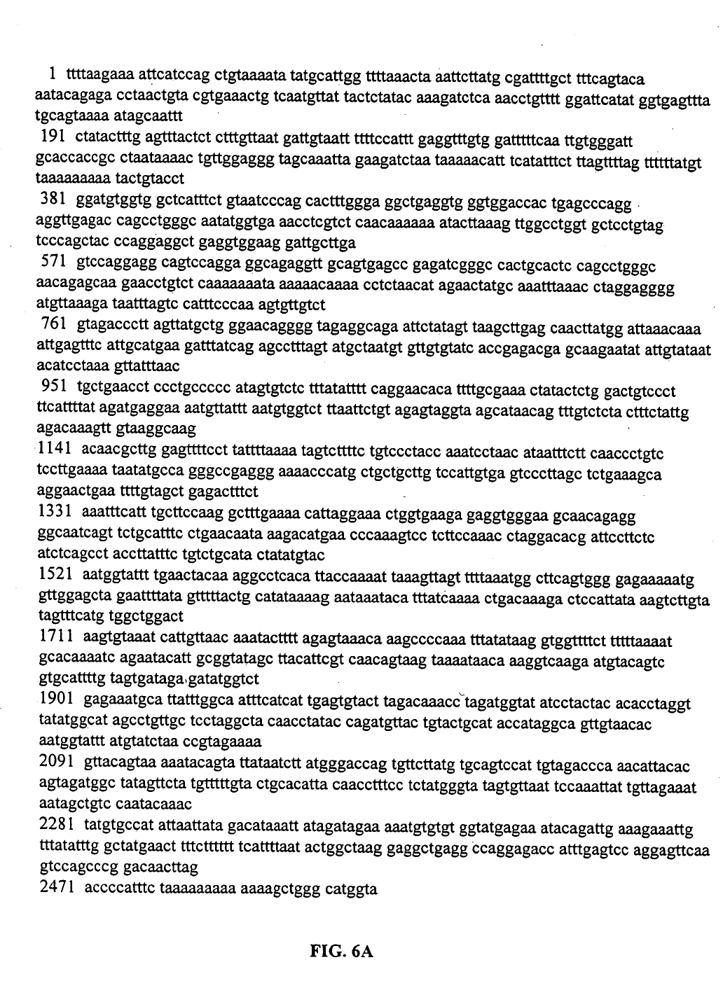

Gene targeting vector depicted in FIG. 5 was constructed as follows. Briefly, sgGFP fragment (http://www.qbiogene.com/protocols/gene-expression/m-afp.pdf (accessed Sep. 5, 2001) was inserted into a tetracycline regulated expression vector pUHD 10-3 (http://www.zmbh.uniheidelberg.de/bujard/homepage.html, accessed Sep. 20, 2001) to generate pTet-GFP. An sgBFP expression cassette was inserted into pTet-GFP as shown in FIG. 5 to generate pGT-GFP/BFP. To target the TSG101 locus, a 4 kb region of TSG101 gene that spans exons 4-6 was chosen (GENEBANK® accession no. NT_{—}009307.5). This 4 kb fragment was divided into homologous recombination region 1 (SEQ ID NO:1) and homologous recombination region 2 (SEQ ID NO:2), each region has about 2 kb in length (see FIGS. 6A-B). Homologous recombination region 1 was inserted into pGT-neo/GFP/BFG at a Hind III site, and homologous recombination region 2 was inserted at an EcoR I site to give pGT-GFP/BFP-TSG111. CLL212-trans cells Cells were transfected with the gene targeting vector (pGT-GFP/BFP-TSG101) by electroporation (Li et al., 1996, Cell 85:319-329). Transfected cells were cultured for 24 to 48 hours. The cell cultures were then trypsinized. Cells were analyzed by FACS. Only cells that expressed GFP but did not express BFP were sorted from the population. The sorted cells were expanded into cell lines. These clones were confirmed to have undergone the desired homologous recombination at the TSG101 locus by genomic Southern blotting analysis and PCR analysis. Western blotting using a rabbit anti-TSG101 antibody (CLONETECH, see also Li et al., Proc. Natl. Acad. Sci. USA, 98:1619-24) further confirmed the inactivation of TSG101 protein production.

### 7. References Cited

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. A method for generating a plurality of cells comprising cells that carry an insertion of a DNA sequence in the genome by homologous recombination, said method comprising transfecting cells of a cell type with a gene targeting vector comprising:
(a) a first sequence region comprising a nucleotide sequence which is substantially homologous to a first target DNA sequence in the genome of cells of said cell type;
(b) a second sequence region comprising a nucleotide sequence which is substantially homologous to a second target DNA sequence in the genome of cells of said cell type;
(c) a third sequence region located between said first and second sequence regions, comprising a nucleotide sequence that encodes a positive selection marker; and
(d) a fourth sequence region comprising a nucleotide sequence encoding a fluorescence marker, located at 5′ to said first or 3′ to said second sequence region, wherein said positive selection marker is expressed in said cells that carry said insertion by homologous recombination, and wherein said fluorescence marker encoded in said fourth sequence region is not expressed in said cells that carry said insertion by homologous recombination.

2. The method of claim 1, wherein said gene targeting vector further comprises a fifth sequence region comprising a DNA sequence encoding a selection marker, wherein said fifth sequence region is located at 5′ to said first sequence region if said fourth sequence region is located at the 3′ to said second sequence region or at 3′ to said second sequence region if said fourth sequence region is located at the 5′ to said first sequence region.

3. The method of claim 1, further comprising the step of selecting said cells that carry said insertion by homologous recombination.

4. The method of claim 3, wherein said step of selecting comprising
(a) selecting cells wherein said positive selection marker is expressed; and
(b) selecting cells wherein said fluorescence marker encoded in said fourth sequence region is not expressed.

5. The method of claim 4, wherein said step (b) is carried out after said step (a).

6. The method of claim 5, wherein said step (b) is carried out by a fluorescence activated cell sorter.

7. The method of claim 1, 2, or 3, wherein said positive selection marker gene is a gene selected from the group consisting of a drug resistance gene, a gene encoding a surface marker, a gene encoding a fluorescence marker, a gene encoding β-galactosidase, and a gene encoding β-geo.

8. The method of claim 5, wherein said positive selection marker gene is a drug resistance gene.

9. The method of claim 8, wherein said drug resistance gene is selected from the group consisting of a Neomycin/G418 resistance gene, a Puromycin resistance gene, a Hygromycin B resistance gene, a Zeocin resistance gene, and a mycophenolic acid resistance gene.

10. The method of claim 4, wherein said positive selection marker gene is a gene encoding a fluorescence marker.

11. The method of claim 10, wherein said gene encoding a fluorescence marker is selected from the group consisting of a gene encoding a green fluorescence marker, a gene encoding a blue fluorescence marker, and a gene encoding a red fluorescence marker.

12. The method of claim 10 or 11, wherein said step (a) is carried out by a fluorescence activated cell sorter.

13. The method of claim 12, wherein said step (a) and step (b) are carried out concurrently.

14. The method of 13, wherein said step of selection is carried out such that said cells that carry said insertion by homologous recombination constitute at least 10% of said plurality of cells.

15. The method of claim 14, wherein said step of selection is carried out such that said cells that carry said insertion by homologous recombination constitute at least 30% of said plurality of cells.

16. The method of claim 15, wherein said step of selection is carried out such that said cells that carry said insertion by homologous recombination constitute at least 50% of said plurality of cells.

17. The method of claim 16, wherein said step of selection is carried out such that said cells that carry said insertion by homologous recombination constitute at least 70% of said plurality of cells.

18. The method of claim 17, wherein said step of selection is carried out such that said cells that carry said insertion by homologous recombination constitute at least 90% of said plurality of cells.

19. The method of any one of claims 3-6 and 8-18, wherein said gene targeting vector further comprises a fifth sequence region comprising a DNA sequence encoding a selection marker, wherein said fifth sequence region is located at 5′ to said first sequence region if said fourth sequence region is located at the 3′ to said second sequence region or at 3′ to said second sequence region if said fourth sequence region is located at the 5′ to said first sequence region, and wherein said method further comprises a step of selecting cells wherein said selection marker encoded in said fifth sequence region is not expressed.

20. The method of claim 19, wherein said selection marker encoded in said fifth sequence region is a fluorescence marker.

21. The method of claim 4 or 5, wherein said positive selection marker gene is a gene encoding a surface marker.

22. The method of claim 4 or 5, wherein said positive selection marker gene is a gene encoding β-galactosidase.

23. The method of claim 4 or 5, wherein said positive selection marker gene is a gene encoding β-geo.

24. The method of any one of claims 1-6, wherein said positive selection marker gene is a gene encoding a combination of more than one selection markers.

25. The method of claim 24, wherein said gene encoding a combination of more than one selection markers encodes a rsGFP-neo fusion protein.

26. The method of claim 24, wherein said gene targeting vector further comprises a fifth sequence region comprising a DNA sequence encoding a selection marker, wherein said fifth sequence region is located at 5′ to said first sequence region if said fourth sequence region is located at the 3′ to said second sequence region or at 3′ to said second sequence region if said fourth sequence region is located at the 5′ to said first sequence region.

27. The method of 5 or 6, wherein said step (b) is carried out such that at least 10% of the sorted cells from the initial cell population are cells that do not carry the insertion of the fluorescence marker gene encoded in the fourth sequence region of the gene targeting vector.

28. The method of claim 27, wherein said step (b) is carried out such that at least 30% of the sorted cells from the initial cell population are cells that do not carry the insertion of the fluorescence marker gene encoded in the fourth sequence region of the gene targeting vector.

29. The method of claim 28, wherein said step (b) is carried out such that at least 50% of the sorted cells from the initial cell population are cells that do not carry the insertion of the fluorescence marker gene encoded in the fourth sequence region of the gene targeting vector.

30. The method of claim 29, wherein said step (b) is carried out such that at least 70% of the sorted cells from the initial cell population are cells that do not carry the insertion of the fluorescence marker gene encoded in the fourth sequence region of the gene targeting vector.

31. The method of claim 30, wherein said step (b) is carried out such that at least 90% of the sorted cells from the initial cell population are cells that do not carry the insertion of the fluorescence marker gene encoded in the fourth sequence region of the gene targeting vector.

32. A gene targeting vector for inserting a DNA sequence in the genome of cells of a cell type, comprising
(a) a first sequence region comprising a nucleotide sequence which is substantially homologous to a first target DNA sequence in the genome of cells of said cell type;
(b) a second sequence region comprising a nucleotide sequence which is substantially homologous to a second target DNA sequence in the genome of cells of said cell type;
(c) a third sequence region located between said first and second sequence regions, comprising a nucleotide sequence that encodes a positive selection marker; and
(d) a fourth sequence region comprising a nucleotide sequence encoding a fluorescence marker, located at 5′ to said first or 3′ to said second sequence region, wherein said positive selection marker is expressed in said cells if said nucleotide sequence encoding said positive selection marker is integrated in the genome of said cells, and wherein said fluorescence marker is expressed in said cells if said nucleotide sequence encoding said fluorescence marker is integrated in the genome of said cells.

33. The gene targeting vector of claim 32, wherein said positive selection marker gene is a drug resistance gene.

34. The gene targeting vector of claim 33, wherein said drug resistance gene is selected from the group consisting of a Neomycin/G418 resistance gene, a Puromycin resistance gene, a Hygromycin B resistance gene, a Zeocin resistance gene, and a mycophenolic acid resistance gene.

35. The gene targeting vector of claim 32, wherein said positive selection marker gene is a gene encoding a fluorescence marker.

36. The gene targeting vector of claim 35, wherein said gene encoding a fluorescence marker is selected from the group consisting of a gene encoding a green fluorescence marker, a gene encoding a blue fluorescence marker, and a gene encoding a red fluorescence marker.

37. The gene targeting vector of claim 32, wherein said positive selection marker gene is a gene encoding a surface marker.

38. The gene targeting vector of claim 32, wherein said positive selection marker ene is a gene encoding β-galactosidase.

39. The gene targeting vector of claim 32, wherein said positive selection marker gene is a gene encoding β-geo.

40. The gene targeting vector of claim 32, wherein said positive selection marker gene is a gene encoding a combination of more than one selection markers.

41. The gene targeting vector of claim 40, wherein said gene encoding a combination of more than one selection markers encodes a rsGFP-neo fusion protein.

42. The gene targeting vector of any one of claims 32-41, wherein said gene encoding a fluorescence marker is selected from the group consisting of a gene encoding a green fluorescence marker, a gene encoding a blue fluorescence marker, and a gene encoding a red fluorescence marker.

43. The gene targeting vector of any one of claims 32-41, further comprising a fifth sequence region comprising a DNA sequence encoding a selection marker, wherein said fifth sequence region is located at 5′ to said first sequence region if said fourth sequence region is located at the 3′ to said second sequence region or at 3′ to said second sequence region if said fourth sequence region is located at the 5′ to said first sequence region.

44. The method of claim 43, wherein said selection marker encoded in said fifth sequence region is a fluorescence marker.

45. The method of claim 44, wherein said fluorescence marker is the same as said fluorescence marker encoded in the fourth sequence region.

46. The method of claim 44, wherein said fluorescence marker is different from said fluorescence marker encoded in the fourth sequence region.
